(19) United States
(12) Patent Application Publication
(10) Pub. No.: US 2002/0058765 A1
(43) Pub. Date: May 16, 2002

(54) Catalyst system for the polymerization of olefins

(76) Inventors: Sigurd Becke, Rosrath (DE); Uwe Denninger, Bergisch Gladbach (DE); Steffen Kahlert, Koln (DE); Werner Obrecht, Moers (DE); Claudia Schmid, Leichlingen (DE); Heike Windisch, Leverkusen (DE)

Correspondence Address: BAYER CORPORATION, PATENT DEPARTMENT, 100 BAYER ROAD, PITTSBURGH, PA, 15205, US

(21) Appl. No.: 09/922,134

(22) Filed: Aug. 3, 2001

(30) Foreign Application Priority Data
Aug. 9, 2000 (DE) 10039626.7
Aug. 21, 2000 (DE) 10040860.5

(51) Int. Cl. C08F 4/44; C07C 43/00

(52) U.S. Cl. 526/160; 526/943; 526/282; 526/335; 526/346; 526/348.2; 526/348.5; 526/348.6; 526/348.7; 556/465; 568/18; 568/631; 568/630; 570/186; 568/579

(57) ABSTRACT

The present invention relates to a metal-free cyclopentadienide compound which, in conjunction with a metallocene, is able to form a catalyst system that can be used for the polymerization of olefins. It is thus possible to dispense with the use of methylaluminoxane (MAO) or boron-containing compounds as co-catalyst and nevertheless achieve a high degree of catalytic activity. The invention relates also to a process for the preparation of the metal-free cyclopentadienide compound and to the use thereof as a catalyst component in the preparation of polyolefins.

### FIELD OF THE INVENTION

The present invention relates to a metal-free cyclopentadienide compound which, in conjunction with a metallocene, is able to form a catalyst system that can be used for the polymerization of olefins. It is, thus, possible to dispense with the use of methylaluminoxane (MAO) or boron-containing compounds as co-catalyst and nevertheless achieve a high degree of catalytic activity. The invention relates also to a process for the preparation of the metal-free cyclopentadienide compound and to the use thereof as a catalyst component in the preparation of polyolefins.

### BACKGROUND OF THE INVENTION

Metal-containing cyclopentadienide compounds have long been known. For example, the compound cyclopentadienyllithium is formed by reaction of cyclopentadiene with butyllithium with formation of the cyclopentadienide anion (Cp anion). In the case of magnesium, the magnesocene is known, in which two Cp anions are bonded to the magnesium. Cp anions form stable complexes with transition metals. In the case of ferrocene, there are two Cp anions, which enclose the metal atom in such a manner that a so-called sandwich compound (metallocene) is formed. The bond between the Cp anion and the transition metal atom is particularly stable in the case of metallocenes, since coordination of the Cp anion takes place via the π electrons of the C₅H₅ ring.

Metallocenes on their own do not have polymerization activity. Active polymerization catalysts are formed in combination with co-catalysts, for example MAO (Macromol. Symp. Vol. 97, July 1995).

However, catalyst systems based on metallocenes and alumoxanes in some cases have considerable disadvantages. For example, alumoxanes, especially MAO, cannot be prepared with a high degree of reproducibility either in situ or in a pre-forming process. MAO is a mixture of various aluminum alkyl-containing species that are present in equilibrium relative to one another, which is at the expense of reproducibility in the polymerization of olefinic compounds. Moreover, MAO is not stable to storage and changes its composition when subjected to heat. A further serious disadvantage is the large excess of MAO that is required in the activation of metallocenes. However, the high MAO/metallocene ratio is a necessary requirement for the achievement of high catalytic activity. It results in a critical process disadvantage, however, since the aluminum compounds must be separated from the polymers during working up. In addition, MAO is a cost-determining factor in the use of MAO-containing catalyst systems, which means that MAO excesses are uneconomical.

In J. Am. Chem. Soc. 1991, 113, 3623, tris(pentafluorophenyl)borane is described as a co-catalyst for metallocene dialkyls. The polymerization activity of catalysts based on tris(pentafluorophenyl)borane is unsatisfactory, however. EP-A1-277 003 and EP-A1-277 004 describe ionic catalyst systems prepared by reaction of metallocenes with ionizing reagents. Perfluorinated tetraaromatic borate compounds, especially tetrakis(pentafluorophenyl) borate compounds, are preferably used as the ionizing reagents (EP-A1-0 468 537, EP-A1-0 561 479). EP-A1-0 561 479 describes a compound of the general formula (I)

[(L′−H⁺]_{d}[(M′)^{m+}Q₁Q₂ . . . Qₙ]^{d-} (I)

in which M is a metal or metalloid of groups V-B to V-A of the PSE of the elements. A disadvantage of that compound is that the bond between M and the radicals Q₁-Q₄ is polarized.

That leads to a weakening of the bond, which may result in the groups Q₁-Q₄ being separated off, which is also described in EP-A1-0 277 004.

Compounds of the type BR₄⁻, such as are described in EP-A1-0 561 479, may dissociate on contact with metallocene dialkyls with removal of an alkyl radical, which is to be regarded as a disadvantage since the co-catalyst is thus destroyed.

### SUMMARY OF THE INVENTION

An object of the present invention was to find a co-catalytically active, thermodynamically stable compound for the metallocene-catalyzed polymerization of unsaturated compounds, with which compound some or all of the disadvantages of the prior art are avoided.

A further object was to provide a catalyst system for olefin polymerization having adequate polymerization activity. In particular, the object was to find a catalyst system suitable for the preparation of EPDM.

It has now been found that cyclopentadienide compounds having bulky substituents are especially suitable for achieving that object.

### DETAILED DESCRIPTION OF THE INVENTION

Accordingly, the present invention relates to a metal- and metalloid-free cyclopentadienide compound of the general formula (II)

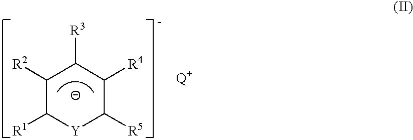

wherein

Q⁺ represents a Lewis-acid cation according to the Lewis acid-base theory (as described, for example, in J. Huheey, Anorganische Chemie, Walter de Gruyter, Berlin, N.Y., 1988 p. 315 f), preferably carbonium, oxonium or/and sulfonium cations, especially the triphenylmethyl cation, or

Q⁺ represents a Brönstedt-acid cation according to the Brönstedt acid-base theory (as described, for example, in J. Huheey, Anorganische Chemie, Walter de Gruyter, Berlin, N.Y., 1988 p. 309 f), preferably trialkylammonium, dialkylarylammonium or/and alkyldiarylammonium, especially N,N-dimethylanilinium,

Y represents a (CR₂⁶)ₘ group wherein m=from 0 to 4, wherein the radicals R⁶ may be identical or different and, when m=0, the ring may be closed or open, with the proviso that, when the ring is open, the free valences at the terminal carbon atoms are saturated by radicals R having the same meaning as R¹-R⁶,

R¹-R⁶ represent identical or different substituents selected from the group of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, silyl optionally substituted by C₁-C₁₀-hydrocarbon radicals, amine substituted by C₁- to C₂₀-hydrocarbon radicals, with the proviso that at least one substituent, preferably at least two substituents, especially at least three substituents, are bulky.

Metalloids denotes the elements boron and aluminum.

Bulky substituents within the scope of the present invention are substituents that make more difficult the formation of a covalent bond between Q⁺ and the cyclopentadienide anion.

Examples, thereof, are branched alkyl groups, mono- or poly-substituted silyl groups, mono- or poly-substituted amino groups, mono- or poly-substituted phosphino groups, aromatic compounds, optionally substituted aromatic compounds, preferably C₁-C₁₀-haloalkyl, C₆-C₂₀-haloaryl, C₆- to C₂₀-aryl, C₆- to C₁₀-alkoxyaryl, especially C₁-C₁₀-fluoroalkyl, C₆-C₂₀-chloroaryl, C₁-C₁₀-chloroalkyl, C₆-C₂₀-fluoroaryl, C₆- to C₂₀-aryl, C₆- to C₁₀-alkoxyaryl, more especially C₁-C₁₀-fluoroalkyl, C₆-C₂₀-fluoroaryl, C₆- to C₂₀-aryl, C₆- to C₁₀-alkoxyaryl.

Special preference is given to compounds of formula II wherein at least one R from R¹-R⁶ represents a halogen-containing, more especially a chlorine- and/or fluorine-containing, aromatic compound of formula VI

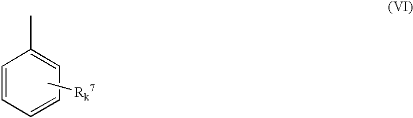

wherein

k represents an integer in the range from 1 to 5, and

R⁷ is selected from the group consisting of C₁-C₂₀-alkyl, C₁-C₂₀-alkoxy, hydrogen, halogen, C₁-C₂₅-haloalkyl, with the proviso that at least one R⁷ represents halogen or C₁-C₂₅-haloalkyl, with fluorine and C₁-C₂₅-fluoroalkyls being very especially preferred.

Examples of substituents of formula VI are 4-fluorophenyl, 4-chlorophenyl, 3-fluorophenyl, 3-chlorophenyl, 2-fluorophenyl, 2-chlorophenyl, 2,6-difluorophenyl, 2,6-dichlorophenyl, 2,4-difluorophenyl, 2,4-dichlorophenyl, 2,3-difluorophenyl, 2,3-dichlorophenyl, 2,5-difluorophenyl, 2,5-dichlorophenyl, 3,4-difluorophenyl, 3,4-dichlorophenyl, 3,5-difluorophenyl, 3,5-dichlorophenyl, 2,4,6-trifluorophenyl, 3,4,5-trifluorophenyl, 2,3,4-trifluorophenyl, 2,3,5-trifluorophenyl, 2,3,6-trifluorophenyl, 2,3,4,5-tetrafluorophenyl, 2,3,5,6-tetrafluorophenyl, 4,5,6-tetrafluorophenyl, pentafluorophenyl, 4-(trifluoromethyl)phenyl, 2,6-bis-(trifluoromethyl)phenyl, 3,5-bis-(trifluoromethyl)phenyl, 3,4,5-tris-(trifluoromethyl)phenyl, 2,4,4-tris-(trifluoromethyl)phenyl, with special preference being given to 4-fluorophenyl, 2,6-difluorophenyl, 2,4-difluorophenyl, 2,4,6-trifluorophenyl, pentafluorophenyl, 3,5-bis-(trifluoromethyl)phenyl and compounds corresponding to the compounds just mentioned in which one or more fluorine atoms have been replaced by chlorine atoms, but the further listing of which would not make any additional contribution towards the understanding of the Application.

The radicals R¹ to R⁶, in each case together with the atoms bonding them, may form one or more aliphatic or aromatic ring systems that may contain one or more hetero atoms selected from the group N, P, Si and that have from 5 to 10 carbon atoms.

There may be mentioned by way of example compounds of formulae IIa and IIb:

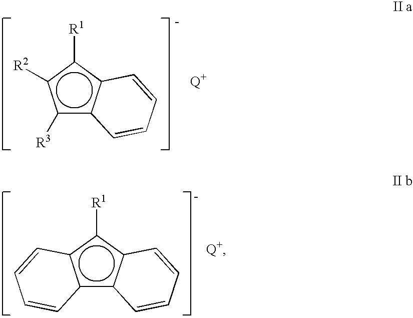

wherein

Q⁺ represents a Lewis-acid cation according to the Lewis acid-base theory (see above), preferably carbonium, oxonium or/and sulfonium cations, especially the triphenylmethyl cation, or

Q⁺ represents a Brönstedt-acid cation according to the Brönstedt acid-base theory (see above), preferably trialkylammonium, dialkylarylammonium or/and alkyldiarylammonium, especially N,N-dimethylanilinium, and

R¹-R³ are as defined in (II).

It goes without saying that the ring systems may in turn be substituted. Suitable substituents are, for example, the examples given for R¹-R⁶.

Especially preferred cyclopentadienide compounds of the general formula (II) are compounds of formula (IIc)

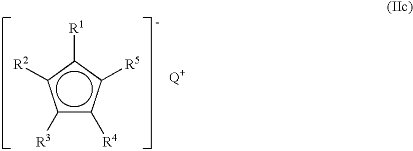

wherein

R¹-R⁵ and Q⁺ are as defined above.

In the preparation of cyclopentadienes, indenes or fluorenes substituted in the 1-position by aryl, the corresponding cyclopentadienones, indenones or fluorenones are expediently used as starting material and are converted into the cyclopentadienes, indenes or fluorenes according to R. H. Lowack and K. P. C. Vollhardt (J. organomet. Chem., 1994, 476, 25-329).

Tetraaryl-substituted cyclopentadienones, if they are not available commercially, can be prepared according to W. Dilthey and F. Quint (J. prakt. Chem. 1930, 128, 139) from benzil derivatives and 1,3-diarylacetones, according to M. Miura, S. Pivsa-Art, G. Dyker, J. Heiermann, T. Satoh, M. Nomura (Cem. Comm. 1998,1889) from zirconocene dichloride and aryl bromides by a Heck reaction, or according to J. M. Birchall, F. L. Bowden, R. N. Hazeldine and A. b. P. Lever (J. Cem. Soc. (A) 1967, 747) by reaction of corresponding tolanes with dicobalt octacarbonyl.

The cyclopentadienes substituted in the 1-position by aryl are obtainable from those cyclopentadienones by reaction with aryllithium or arylmagnesium halides at low temperatures and subsequent reduction with zinc/acetic acid, lithium alanate or other suitable reducing agents.

The preparation of the metal-free cyclopentadienide compound according to the present invention forms a further subject of the present invention and is preferably carried out by replacing a proton from a corresponding diene compound, preferably a cyclopentadiene, in which the substituents R¹-R⁶ are as defined above, by a metal or an organometallic compound, preferably an alkali metal or an organometallic compound of group 1, 12 or 14.

Next, a reaction follows with an alkyl metal compound or a metal, preferably an alkyl alkali metal compound or an alkali metal or an organometallic compound of group 12 or 14. n-Butyllithium, tert-butyllithium, sodium and potassium have proven to be especially suitable.

Then, a reaction follows with a halide of the corresponding metal- and metalloid-free cation, in order to obtain the compounds (II).

Reaction with dimethylanilinium hydrochloride or tritylium chloride has proven to be especially advantageous. Suitable solvents for the formation of the compounds according to the present invention are aliphatic and aromatic hydrocarbons, ethers and cyclic ethers. Examples thereof are pentane, hexane, heptane, octane, cyclohexane, benzene, toluene, xylene, dialkyl ethers and tetrahydrofuran. Mixtures of different solvents are also suitable.

The synthesis of the compounds according to the present invention is simple to carry out even on an industrial scale. Due to its crystallizing power, the substances can be prepared in high purity and in good yields. In order to purify them, it is simply necessary to remove the metal halide that forms in the reaction, which is readily possible due to its poor solubility in hydrocarbons.

It has also been found that the metal-free cyclopentadienide compounds according to the present invention are suitable for the preparation of a catalyst system for the polymerization of olefins. Accordingly, the invention also provides catalyst systems containing

a) at least one cyclopentadienide compound according to the present invention

b) at least one organic transition metal compound

and, optionally, additionally containing an organoaluminum compound.

There are suitable as aluminum compounds that may optionally be present, especially trialkylaluminum compounds, dialkylaluminum hydrides, dialkylaluminum chlorides, and alkylaluminum dichlorides. Preferable aluminum compounds are trimethylaluminum, triethylaluminum, triisobutylaluminum, triisooctylaluminum, diisobutylaluminum hydride, and diethylaluminum chloride.

Metallocene compounds, for example, are used as the organic transition metal compound. They may be, for example, bridged or unbridged biscyclopentadienyl complexes, as are described, for example, in EP-A1-0 129 368, EP-A1-0 561 479, EP-A1-0 545 304 and EP-A1-0 576 970, monocyclopentadienyl complexes, such as bridged aminocyclopentadienyl complexes, which are described, for example, in U.S. Pat. No. 5,721,185, polynuclear cyclopentadienyl complexes as described in EP-A1-0 632 063, π-ligand-substituted tetrahydropentalenes as described in EP-A1-0 661 300. It is also possible to use organometallic compounds in which the complexing ligand does not contain cyclopentadienyl ligands. Examples thereof are diamine complexes of groups 3 and 4 of the periodic system of the elements according to IUPAC 1986, such as are described, for example, in D. H. McConville et al., Macromolecules, 1996, 29, 5241 and in D. H. McConville et al., J. Am. Chem. Soc., 1996, 118, 10008. It is also possible to use diimine complexes of sub-group VIII of the periodic system of the elements (e.g. Ni²⁺ or Pd²⁺ complexes), as are described in Brookhart etal., J. Am. Chem. Soc., 1995, 117, 6414 and Brookhart et al., J. Am. Chem. Soc., 1996, 118, 267.

It is also possible to use 2,6-bis(imino)pyridyl complexes of groups 8-10 of the periodic system of the elements (e.g. Co²⁺ or Fe²⁺ complexes), such as are described in Brookhart et al., J. Am. Chem. Soc., 1998, 120, 4049 and in Gibson et al., Chem. Comm. 1998, 849. For the purposes of US patent practice, the above-mentioned documents are incorporated by reference in the present Application.

Preferred metallocene compounds are unbridged and bridged compounds of formula IV

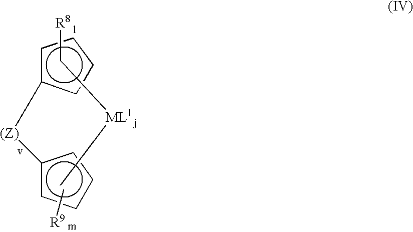

wherein

M is a metal of groups 3-6 of the periodic system of the elements according to IUPAC 1986, especially Ti, Zr or Hf,

R⁸ represents one or more identical or different substituents and is/are selected from hydrogen, SiR¹⁰₃, BR¹⁰₂ or PR¹⁰₂, wherein the radicals R¹⁰ are identical or different and are selected from hydrogen or a group containing from 1 to 40 carbon atoms, preferably a C₁-C₂₀-alkyl group, a C₁-C₁₀-haloalkyl group, a C₆-C₁₀-haloaryl group, a C₁-C₁₀-alkoxy group, a C₆-C₂₀-aryl group, a C₆-C₁₀-aryloxy group, a C₂-C₁₀-alkenyl group, a C₇-C₄₀-arylalkyl group, a C₈-C₄₀-arylalkenyl group, a C₂-C₁₀-alkynyl group, a C₆-C₂₄-heteroaryl group such as pyridyl, furyl or quinolyl, preference being given in the case of halogen compounds to fluorine and chlorine, more preferably fluorine.

Where a plurality of radicals R⁸ is present, two or more radicals R⁸ may be bonded together in such a manner that those radicals R⁸ and the atoms of the cyclopentadienyl ring bonding them form a C₄ to C₂₄ ring system, which may in turn be substituted,

R⁹ represents one or more identical or different substituents and is/are selected from the group consisting of hydrogen atom, SiR¹¹₃, BR¹¹₂ or PR¹¹₂, wherein the radicals R¹¹ are identical or different and are selected from the group consisting of hydrogen or a group containing from 1 to 40 carbon atoms, preferably a C₁-C₂₀-alkyl group, a C₁-C₁₀-haloalkyl group, a C₆-C₁₀-haloaryl group, a C₁-C₁₀-alkoxy group, a C₆-C₂₀-aryl group, a C₆-C₁₀-aryloxy group, a C₂-C₁₀-alkenyl group, a C₇-C₄₀-arylalkyl group, a C₈-C₄₀-arylalkenyl group, a C₂-C₁₀-alkynyl group, a C₆-C₂₄-heteroaryl group such as pyridyl, furyl or quinolyl, preference being given in the case of halogen compounds to fluorine and chlorine, more preferably fluorine.

Where a plurality of radicals R⁹ is present, two or more radicals R⁹ may be bonded together in such a manner that those radicals R⁹ and the atoms of the cyclopentadienyl ring bonding them form a C₄ to C₂₄ ring system, which may in turn be substituted,

l is 5 when v=0 and

l is 4 when v=1,

m is 5 when v=0 and

m is 4 when v=1,

L¹ may be identical or different and is/are selected from the group consisting of hydrogen, a C₁-C₁₀ hydrocarbon group, such as C₁- to C₁₀-alkyl or C₆- to C₁₀-aryl, halogen or OR¹², SR¹², OSiR¹²₃, SiR¹²₃, PR¹²₂, NR¹²₂, wherein R¹² is a halogen atom, a C₁- to C₁₀-alkyl group or a C₆- to C₁₀-aryl group, a halogenated C₁- to C₁₀-alkyl group or a halogenated C₆- to C₁₀-aryl group, or

L¹ represents a toluenesulfonyl, trifluoroacetyl, trifluoroacetoxyl, trifluoromethanesulfonyl, nonafluorobutane-sulfonyl or 2,2,2-trifluoroethanesulfonyl group,

j is an integer from 1 to 4, preferably 2,

Z denotes a bridging structural element between the two cyclopentadienyl rings and v is 0 or 1, wherein the bridge may be of covalent or coordinative nature.

Examples of Z are groups M²R¹³R¹⁴, wherein M² is carbon, silicon, germanium or tin and R¹³ and R¹⁴ are identical or different and represent a C₁-C₂₀ hydrocarbon-containing group, such as C₁- to C₁₀-alkyl or C₆- to C₁₄-aryl or trimethylsilyl. Z is preferably CH₂, CH₂CH₂, CH(CH₃)CH₂, CH(C₄H₉)C(CH₃)₂, C(CH₃)₂,-Si(CH₃)₂, Ge(CH₃)₂, Sn(CH₃)₂,(C₆H₅)2Si, (C₆H₅)(CH₃)Si, (C₆H₅)₂Ge, (C₆H₅)₂Sn, (CH₂)₄Si, CH₂Si(CH₃)₂, o—C₆H₄ or 2,2′-(C₆H₄)₂. Z may also form a mono- or poly-cyclic ring system with one or more radicals R¹² and/or R¹³.

Preference is given to chiral bridged metallocene compounds of formula IV, especially those in which v is 1 and one or both of the cyclopentadienyl rings are so substituted that they represent an indenyl ring. The indenyl ring is in that case preferably substituted, especially in the 2-, 4-, 2,4,5-, 2,4,6-, 2,4,7- or 2,4,5,6-position, by C₁-C₂₀ hydrocarbon-containing groups, such as C₁- to C₁₀-alkyl or C₆- to C₁₄-aryl, it also being possible for two or more substituents of the indenyl ring together to form a ring system.

Chiral bridged metallocene compounds of formula IV may be used in the form of pure racemic or pure meso compounds. However, it is also possible to use mixtures of a racemic compound and a meso compound.

Preferred examples of metallocene compounds of formula (IV) are:

dimethylsilanediylbis(indenyl)zirconium dichloride

dimethylsilanediylbis(4-naphthyl-indenyl)zirconium dichloride

dimethylsilanediylbis(2-methyl-benzo-indenyl)zirconium dichloride

dimethylsilanediylbis(2-methyl-indenyl)zirconium dichloride

dimethylsilanediylbis(2-methyl-4-(1-naphthyl)-indenyl)zirconium dichloride

dimethylsilanediylbis(2-methyl-4-(2-naphthyl )-indenyl)zirconium dichloride

dimethylsilanediylbis(2-methyl-4-phenyl-indenyl)zirconium dichloride

dimethylsilanediylbis(2-methyl-4-tert-butyl-indenyl)zirconium dichloride

dimethylsilanediylbis(2-methyl-4-isopropyl-indenyl)zirconium dichloride

dimethylsilanediylbis(2-methyl-4-ethyl-indenyl)zirconium dichloride

dimethylsilanediylbis(2-methyl-4-acenaphth-indenyl)zirconium dichloride

dimethylsilanediylbis(2,4-dimethyl-indenyl)zirconium dichloride

dimethylsilanediylbis(2-ethyl-indenyl)zirconium dichloride

dimethylsilanediylbis(2-ethyl-4-ethyl-indenyl)zirconium dichloride

dimethylsilanediylbis(2-ethyl-4-phenyl-indenyl)zirconium dichloride

dimethylsilanediylbis(2-methyl-4,5-benzo-indenyl)zirconium dichloride

dimethylsilanediylbis(2-methyl-4,6-diisopropyl-indenyl)zirconium dichloride

dimethylsilanediylbis(2-methyl-4,5-diisopropyl-indenyl)zirconium dichloride

dimethylsilanediylbis(2,4,6-trimethyl-indenyl)zirconium dichloride

dimethylsilanediylbis(2,5,6-trimethyl-indenyl)zirconium dichloride

dimethylsilanediylbis(2,4,7-trimethyl-indenyl)zirconium dichloride

dimethylsilanediylbis(2-methyl-5-isobutyl-indenyl)zirconium dichloride

dimethylsilanediylbis(2-methyl-5-tert-butyl-indenyl)zirconium dichloride

methyl(phenyl)silanediylbis(2-methyl-4-phenyl-indenyl)zirconium dichloride

methyl(phenyl)silanediylbis(2-methyl-4,6-diisopropyl-indenyl)zirconium dichloride

methyl(phenyl)silanediylbis(2-methyl-4-isopropyl-indenyl)zirconium dichloride

methyl(phenyl)silanediylbis(2-methyl-4,5-benzo-indenyl)zirconium dichloride

methyl(phenyl)silanediylbis(2-methyl-4,5-(methylbenzo)-indenyl)zirconium dichloride

methyl(phenyl)silanediylbis(2-methyl-4 ,5-(tetramethylbenzo)-indenyl)zirconium dichloride

methyl(phenyl)silanediylbis(2-methyl-4-acenaphth-indenyl)zirconium dichloride

methyl(phenyl)silanediylbis(2-methyl-indenyl)zirconium dichloride

methyl(phenyl)silanediylbis(2-methyl-5-isobutyl-indenyl)zirconium dichloride

1,2-ethanediylbis(2-methyl-4-phenyl-indenyl)zirconium dichloride

1,4-butanediylbis(2-methyl-4-phenyl-indenyl)zirconium dichloride

1,2-ethanediylbis(2-methyl-4,6-diisopropyl-indenyl)zirconium dichloride

1,4-butanediylbis(2-methyl-4-isopropyl-indenyl)zirconium dichloride

1,4-butanediylbis(2-methyl-4,5-benzo-indenyl)zirconium dichloride

1,2-ethaned iylbis(2-methyl-4,5-benzo-indenyl)zirconium dichloride

1,2-ethanediylbis(2,4,7-trimethyl-indenyl)zirconium dichloride

1,2-ethanediylbis(2-methyl-indenyl)zirconium dichloride

1,4-butanediylbis(2-methyl-indenyl)zirconium dichloride

[4-(η⁵-cyclopentadienyl)-4,6,6-trimethyl-(η⁵-4,5-tetrahydropentalene)]-dichlorozirconium

[4-(η⁵-3′-trimethylsilyi-cyclopentadienyl)-4,6,6-trimethyl-(η⁵-4,5-tetrahydro-pentalene)]dichlorozirconium

[4-(η⁵-3′-isopropyl-cyclopentadienyl)-4,6,6-trimethyl-(η⁵-4,5-tetrahydropentalene)]-dichlorozirconium

[4-(η⁵-cyclopentadienyl)-4,7,7-trimethyl-(η⁵-4,5,6,7-tetrahydroindenyl)]-dichlorotitanium

[4-(η⁵-cyclopentadienyl)-4,7,7-trimethyl-(η⁵-4,5,6,7-tetrahydroindenyl)]-dichlorozirconium

[4-(η⁵-cyclopentadienyl)-4,7,7-trimethyl-(η⁵-4,5,6,7-tetrahydroindenyl)]-dichlorohafnium

[4-(η⁵-3′-tert-butyl-cyclopentadienyl)-4,7,7-trimethyl-(η⁵-4,5,6,7-tetrahydro-indenyl)]-dichlorotitanium

4-(η⁵-3′-isopropylcyclopentadienyl)-4,7,7-trimethyl-(η⁵-4,5,6,7-tetrahydro-indenyl)]-dichlorotitanium

4-(η⁵-3′-methyicyclopentadienyl)-4,7,7-trimethyl-(η⁵-4,5,6,7-tetrahydro-indenyl)]-dichlorotitanium

4-(η⁵-3′-trimethylsilyl-cyclopentadienyl)-2-trimethylsilyl-4,7,7-trimethyl-(η⁵-4,5,6,7-tetrahydroindenyl)]-dichlorotitanium

4-(η⁵-3′-tert-butyl-cyclopentadienyl)-4,7,7-trimethyl-(η⁵-4,5,6,7-tetrahydro-indenyl)]-dichlorozirconium

(tert-butylamido)-(tetramethyl-η⁵-cyclopentadienyl)-dimethylsilyl-dichlorotitanium

(tert-butylamido)-(tetramethyl-η⁵-cyclopentadienyl)-1,2-ethanediyl-dichlorotitanium

(methylamido)-(tetramethyl-η⁵-cyclopentadienyl)-dimethylsilyl-dichlorotitanium

(methylamido)-(tetramethyl-η⁵-cyclopentadienyl)-1,2-ethanediyl-dichlorotitanium

(tert-butylamido)-(2,4-dimethyl-2,4-pentadien-1-yl)-dimethylsilyl-dichlorotitanium

bis-(cyclopentadienyl)-zirconium dichloride

bis-(n-butylcyclopentadienyl)-zirconium dichloride

bis-(1,3-dimethylcyclopentadienyl)-zirconium dichloride

tetrachloro-[1-[bis(η⁵-1H-inden-1-ylidene)methylsilyl]-3,η⁵-cyclopenta-2,4-dien-1-ylidene)-3-η⁵-9H-fluoren-9-ylidene)butane]di-zirconium

tetrachloro-[(η⁵-cyclopentadienyl)-4,6,6-trimethyl-(η⁵-4,5-tetrahydropentalene)]dichlorozirconium

[4-(η⁵-3′-trimethylsilyl-cyclopentadienyl)-4,6,6-trimethyl-(η⁵-4,5-tetrahydro-pentalene)]-dichlorozirconium

[4-(η⁵-3′-isopropyl-cyclopentadienyl)-4,6,6-trimethyl-(η⁵-4,5-tetrahydropentalene)]-dichlorozirconium

[4-(η⁵-cyclopentadienyl)-4,7,7-trimethyl- (η⁵-4,5,6,7-tetrahydroindenyl)]-dichlorotitanium

[4-(η⁵-cyclopentadienyl)-4,7,7-trimethyl-(η⁵-4,5,6,7-tetrahydroindenyl)]-dichlorozirconium

[4-(η⁵-cyclopentadienyl)-4,7,7-trimethyl-(η⁵-4,5,6,7-tetrahyd roindenyl)]-dichlorohafnium

[4-(η⁵-3′-tert-butyl-cyclopentadienyl)-4,7,7-trimethyl(η⁵-4,5,6,7-tetrahydro-indenyl)]-dichlorotitanium

4-(η⁵-3′-isopropylcyclopentadienyl)-4,7,7-trimethyl(η⁵-4,5,6,7-tetrahydro-indenyl)]-dichlorotitanium

4-(η⁵-3′-methylcyclopentadienyl)-4,7,7-trimethyl-(η⁵-4,5,6,7-tetrahydro-indenyl)]-dichlorotitanium

4-(η⁵-3′-trimethylsilyl-cyclopentadienyl)-2-trimethylsilyl-4,7,7-trimethyl-(η⁵-4,5,6,7-tetrahydroindenyl)]-dichlorotitanium

4-(η⁵-3′-tert-butyl-cyclopentadienyl)-4,7,7-trimethyl-(η⁵-4,5,6,7-tetrahydro-indenyl)]-dichlorozirconium

(tert-butylamido)-(tetramethyl-η⁵-cyclopentadienyl)-dimethylsilyl-dichlorotitanium

(tert-butylamido)-(tetramethyl-η⁵-cyclopentadienyl)-1,2-ethanediyl-dichlorotitanium

(methylamido)-(tetramethyl-η⁵-cyclopentadienyl)-dimethylsilyl-dichlorotitanium

(methylamido)-(tetramethyl-η⁵-cyclopentadienyl)-1,2-ethanediyl-dichlorotitanium

(tert-butylamido)-(2,4-dimethyl-2,4-pentadien-1-yl)-dimethylsilyl-dichlorotitanium

bis-(cyclopentadienyl)-zirconium dichloride

bis-(n-butylcyclopentadienyl)-zirconium dichloride

bis-(1,3-dimethylcyclopentadienyl)-zirconium dichloride

tetrachloro-[1-[bis(η⁵-1H-inden-1-ylidene)methylsilyl]-3-,η⁵-cyclopenta-2,4-dien-1-ylidene)-3-η⁵-9H-fluoren-9-ylidene)butane]di-zirconium

tetrachloro-[2-[bis(η⁵-2-methyl-1H-inden-1-ylidene)methoxysilyl]-5-(η⁵-2,3,4,5-tetramethylcyclopenta-2,4-dien-1-ylidene)-5-(η⁵-9H-fluoren-9-ylidene)hexane]di-zirconium

tetrachloro-[1-[bis(η⁵-1H-inden-1-ylidene)methylsilyl]-6-(η⁵-cyclopenta-2,4-dien-1-ylidene)-6-(η⁵-9H-fluoren-9-ylidene)-3-oxaheptane]di-zirconium

dimethylsilanediylbis(2-methyl-4-(tert-butyl-phenyl-indenyl)-zirconium dichloride

dimethylsilanediylbis(2-methyl-4-(4-methyl-phenyl-indenyl)zirconium dichloride

dimethylsilanediylbis(2-methyl-4-(4-ethyl-phenyl-indenyl)zirconium dichloride

dimethylsilanediylbis(2-methyl-4-(4-trifluoromethyl-phenyl-indenyl)zirconium dichloride

dimethylsilanediylbis(2-methyl-4-(4-methoxy-phenyl-indenyl)zirconium dichloride

dimethylsilanediylbis(2-ethyl-4-(4-tert-butyl-phenyl-indenyl)zirconium dichloride

dimethylsilanediylbis(2-ethyl-4-(4-methyl-phenyl-indenyl)zirconium dichloride

dimethylsilanediylbis(2-ethyl-4-(4-ethyl-phenyl-indenyl)zirconium dichloride

dimethylsilanediylbis(2-ethyl-4-(4-trifluoromethyl-phenyl-indenyl)zirconium dichloride

dimethylsilanediylbis(2-ethyl-4-(4-methoxy-phenyl-indenyl)zirconium dichloride

dimethylsilanediylbis(2-methyl-4-(4-tert-butyl-phenyl-indenyl)zirconium dimethyl

dimethylsilanediylbis(2-methyl-4-(4-methyl-phenyl-indenyl)zirconium dimethyl

dimethylsilanediylbis(2-methyl-4-(4-ethyl-phenyl-indenyl)zirconium dimethyl

dimethylsilanediylbis(2-methyl-4-(4-trifluoromethyl-phenyl-indenyl)zirconium dimethyl

dimethylsilanediylbis(2-methyl-4-(4-methoxy-phenyl-indenyl)zirconium dimethyl

dimethylsilanediylbis(2-ethyl-4-(4-tert-butyl-phenyl-indenyl)zirconium dimethyl

dimethylsilanediylbis(2-ethyl-4-(4-methyl-phenyl-indenyl)zirconium dimethyl

dimethylsilanediylbis(2-ethyl-4-(4-ethyl-phenyl-indenyl)zirconium diethyl

dimethylsilanediylbis(2-ethyl-4-(4-trifluoromethyl-phenyl-indenyl)zirconium dimethyl

dimethylsilanediylbis(2-ethyl-4-(4-methoxy-phenyl-indenyl)zirconium dimethyl

The catalyst system can be used for both the homogeneous and the heterogeneous polymerization of olefins. In the case of heterogeneous polymerization, a support material is additionally used, which has optionally been pre-treated.

A process for the preparation of polyolefins in the presence of the catalyst system according to the present invention also forms part of the present invention.

There are polymerized preferably olefins of the formula R^{a}—CH═CH—R^{b}, wherein R^{a} and R^{b} are identical or different and represent a hydrogen atom, a halogen atom, an alkoxy, hydroxy, alkylhydroxy, aldehyde, carboxylic acid or carboxylic acid ester group or a saturated or unsaturated hydrocarbon radical having from 1 to 20 carbon atoms, especially from 1 to 10 carbon atoms, that may be substituted by an alkoxy, hydroxy, alkylhydroxy, aldehyde, carboxylic acid or carboxylic acid ester group, or R^{a} and R^{b}, with the atoms bonding them, form one or more rings. Examples of such olefins are 1-olefins such as ethylene, propylene, but-1-ene, pent-1-ene, 4-methylpent-1-ene, hex-1-ene, oct-1-ene, isobutylene, styrene, cyclic olefins such as norbornene, tetracyclododecene, unconjugated dienes such as vinylnorbornene, ethynylnorbornene or 1,4-hexadiene, methyloctadiene, biscyclopentadiene or methacrylic acid methyl ester.

In particular, the following starting materials are polymerized:

Propylene or ethylene are homopolymerized.

Ethylene is copolymerized with one or more C₃-C₂₀-1-olefins, especially propylene.

Ethylene is copolymerized with one or more C₃-C₂₀-1-olefins, especially propylene, and/or with one or more unconjugated C₄-C₂₀-dienes, especially ethynyinorbornene, vinyinorbornene, 1,4-hexadiene, dicyclopentadiene, methyloctadiene.

Ethylene and cyclic olefins, such as norbornene, are copolymerized.

It may be advantageous to apply the catalyst system according to the invention to a support.

Support materials are used, such as preferably particulate, organic or inorganic solids, the pore volume of which is from 0.1 to 15 ml/g, preferably from 0.25 to 5 ml/g, the specific surface area of which is greater than 1 m²/g, preferably from 10 to 1000 m²/g (BET), the particle size of which is from 10 to 2500 μm, preferably from 50 to 1000 μm, and which may be suitably modified at their surface.

The specific surface area is determined in the conventional manner according to DIN 66 131, the pore volume by the centrifugation method according to McDaniel, J. Colloid Interface Sci. 1980, 78, 31, and the particle size according to Cornillaut, Appl. Opt. 1972, 11, 265.

The following may be mentioned as examples of suitable inorganic solids: silica gels, precipitated silicas, clays, alumosilicates, talcum, zeolites, carbon black, inorganic oxides, such as, for example, silicon dioxide, aluminum oxide, magnesium oxide, titanium dioxide, inorganic chlorides, such as, for example, magnesium chloride, sodium chloride, lithium chloride, calcium chloride, zinc chloride, or calcium carbonate.

The mentioned inorganic solids, which meet the above-mentioned specification and therefore are especially suitable for use as support materials, are described in greater detail, for example, in Ullmanns Enzyklopädie der technischen Chemie, Volume 21, p. 439 ff (silica gels), Volume 23, p. 311 ff (clays), Volume 14, p. 633 ff (carbon blacks) and Volume 24, p. 575 ff (zeolites).

There are suitable as organic solids pulverulent polymeric materials, preferably in the form of free-flowing powders, having the above-mentioned properties. The following may be mentioned as examples, without intending to limit the present invention: polyolefins, such as, for example, polyethene, polypropene, polystyrene, polystyrene-co-divinylbenzene, polybutadiene, polyethers, such as, for example, poly-ethyleneylene oxide, polyoxytetramethylene or polysulfides, such as, for example, poly-p-phenylene sulfide. Especially suitable materials are polypropylene, polystyrene or polystyrene-co-divinylbenzene.

The mentioned organic solids, which meet the above-mentioned specification and therefore, are especially suitable for use as support materials, are described in greater detail, for example, in Ullmanns Enzyklopädie der technischen Chemie, Volume 19, p. 195 ff (polypropylene) and Volume 19, p. 265 ff (polystyrene).

The preparation of the supported catalyst system may be carried out within a wide temperature range. In general, the temperature is between the melting point and the boiling point of the inert solvent mixture. The operation is usually carried out at temperatures of from −50 to +200° C., preferably from −20 to 100° C., especially from 20 to 60° C.

Due to its excellent stability in solution, the catalyst system according to the present invention can be used especially successfully in an industrial Konti process in the solution process.

The invention is explained in greater detail with reference to the examples which follow.

### EXAMPLES

General Information

The preparation and handling of organometallic compounds was carried out with the exclusion of air and moisture under an argon atmosphere (Schlenk technique). All the required solvents were rendered absolute prior to use by boiling for several hours over a suitable drying agent and subsequent distillation under argon. The compounds were characterized by ¹H—NMR, ¹³C—NMR and ¹⁹F—NMR. Other commercial starting materials were used without further purification.

The following substances were obtained commercially:

from Witco: triisobutylaluminum (TIBA), ethylenebis-(tetrahydroindenyl)zirconium dichloride;

from Messer Griesheim GmbH: ethylene, propylene (purity 3.5);

Polymer characterization: The determination of the polymer composition by IR spectroscopy was carried out according to ASTM D 3900.

### Example 1

Preparation of Diiodoacetylene

In a one-liter round-bottomed flask equipped with a dropping funnel and a gas outlet pipe, a constant stream of acetylene is produced by the dropwise addition of water to 380 g of calcium carbide. A second flask having a capacity of 2 liters is filled with a solution of 80 g of potassium iodide in 200 ml of water and 400 ml of 1N sodium hydroxide solution in water. While cooling with ice and with stirring, the acetylene produced in the first flask is introduced into the second flask, while at the same time an approximately 1 N freshly prepared sodium hypochloride solution is added dropwise. During the dropwise addition, a yellow coloring forms at first but quickly disappears, whereupon white diiodoacetylene precipitates. Hypochloride is added dropwise until the initial yellow coloring no longer occurs. The resulting diiodoacetylene is filtered off and then dried in a desiccator over phosphorus(V) oxide. After drying, 42 g of diiodoacetylene are obtained.

### Example 2

Preparation of Decafluorotolane

In a one-liter three-necked flask having a stirrer, a dropping funnel and a reflux condenser, 7.37 g of magnesium chips are covered with a layer of 100 ml of diethyl ether. 74.09 g of bromopentafluorobenzene were slowly added dropwise to that mixture with continuous stirring. When the dropwise addition was complete, the mixture was heated to simmering by means of a water bath and boiled at reflux for about 30 minutes until no more magnesium chips remained. After cooling to room temperature, 300 ml of diethyl ether and then 2.79 g of dried cobalt(II) chloride are added. 41.67 g of diiodoacetylene dissolved in 300 ml of diethyl ether are then slowly added dropwise at a temperature of −20° C. with vigorous stirring, and stirring is then carried out for a further 2 hours. The mixture is then heated to room temperature, and 20% acetic acid is added. After washing 4 times with 300 ml of water, the organic phase is dried over magnesium sulfate. After removal of the solvent in vacuo, the crude product is recrystallized from n-hexane. 23.5 g of decafluorotolane are obtained.

### Example 3

Preparation of 2,3,4,5-Tetrakis(Pentafluorophenyl)Cyclopentadienone

23.21 g of dicobalt octacarbonyl are dissolved in 200 ml of decalin. A total of 23.4 g of decafluorotolane is added in portions to the solution, with vigorous stirring. The solution is then stirred for 5 hours at room temperature and then for 16 hours at 190° C. The decalin is removed in vacuo and the crude product is taken up in a large amount of methylene chloride and introduced into a column packed with aluminum oxide. The product is washed out of the column with approximately 8 liters of methylene chloride. After removal of the solvent in vacuo, 26.24 g of product are obtained in the form of an orange powder.

### Example 4

Preparation of 1,2,3,4,5-Pentakis(Pentafluoro)Phenylcyclopentadiene

2.7 g of pentafluorobenzene are dissolved in 50 ml of diethyl ether and cooled to −70° C. 10 ml of a 1.6 molar solution of n-butyllithium in hexane are then slowly added dropwise, during which the temperature must not exceed −55° C. After the dropwise addition, stirring is carried out for a further 3 hours at −70° C. A solution of 9.67 g of 2,3,4,5-tetrakis(pentafluorophenyl)cyclopentadienone in 80 ml of THF is slowly added dropwise to the resulting solution in such a manner that the temperature does not exceed −70° C. When the addition is complete, stirring is carried out for a further 2 hours at that temperature, followed by heating to room temperature and boiling at reflux for 30 minutes. After removal of the solvent by distillation, 15 ml of glacial acetic acid and 4.5 ml of 48% HBr are added to the residue. The suspension is boiled at reflux for 30 minutes, and then a further 3.5 ml of 48% HBr are added. 3.5 g of zinc dust are then added in small portions for the purposes of reduction. After drying the crude product in vacuo, recrystallization from methylene chloride is carried out. 6.7 g of 1,2,3,4,5-pentakis(pentafluoro)-phenylcyclopentadiene are obtained in the form of a light-yellow microcrystalline solid.

### Example 5

Preparation of 1-Pentafluorophenyl-2,3-Diphenylindene

5.95 g of pentafluorobenzene are dissolved in 50 ml of diethyl ether and cooled to −70° C. 22.1 ml of a 1.6 molar solution of n-butyllithium in hexane are then slowly added dropwise, during which the temperature must not exceed −55° C. After the dropwise addition, stirring is carried out for a further 3 hours at −70° C. A solution of 10 g of 2,3-diphenylindone in 50 ml of THF is slowly added dropwise to the resulting solution in such a manner that the temperature does not exceed −70° C. When the addition is complete, stirring is carried out for a further 2 hours at that temperature, followed by heating to room temperature and boiling at reflux for 30 minutes. After removal of the solvent by distillation, 40.8 ml of glacial acetic acid and 12.8 ml of 48% HBr are added to the residue. The suspension is boiled at reflux for 30 minutes, and then a further 9.7 ml of 48% HBr are added. 9.6 g of zinc dust are then added in small portions for the purposes of reduction. After drying the crude product in vacuo, recrystallization from methylene chloride is carried out. 8.3 g of 1-pentafluorophenyl-2,3-diphenylindene are obtained in the form of a crystalline solid.

### Example 6

Preparation of 1-Pentafluorophenylfluorene

9.33 g of pentafluorobenzene are dissolved in 50 ml of diethyl ether and cooled to −70° C. 35 ml of a 1.6 molar solution of n-butyllithium in hexane are then slowly added dropwise, during which the temperature must not exceed −55° C. After the dropwise addition, stirring is carried out for a further 3 hours at −70° C. A solution of 10 g of fluorenone in 80 ml of THF is slowly added dropwise to the resulting solution in such a manner that the temperature does not exceed −70° C. When the addition is complete, stirring is carried out for a further 2 hours at that temperature, followed by heating to room temperature and boiling at reflux for 30 minutes. After removal of the solvent by distillation, 64 ml of glacial acetic acid and 19 ml of 48% HBr are added to the residue. The suspension is boiled at reflux for 30 minutes, and then a further 15 ml of 48% HBr are added. 15 g of zinc dust are then added in small portions for the purposes of reduction. After drying the crude product in vacuo, recrystallization from methylene chloride is carried out. 7.7 g of 1-pentafluorophenylfluorene are obtained in the form of a microcrystalline solid.

### Example 7

Preparation of Triphenylmethylium 1,2,3,4-Tetraphenyl-5-Pentafluorophenylcyclopentadienide

2 g of 1,2,3,4-tetraphenyl-5-pentafluorophenylcyclopentadiene are dissolved in 20 ml of dried THF and reacted at −78° C. with 2.8 ml of a 1.6 molar solution of n-butyllithium in hexane. After the addition of the n-butyllithium, the reaction mixture is stirred for a further 4 hours at room temperature. The reaction mixture is then cooled to 0° C. and reacted with 1.25 g of triphenylmethyl chloride. After heating to room temperature, stirring is again carried out for 4 hours. After removal of the solvent in vacuo, the crude product is taken up in 20 ml of dried diethyl ether and filtered over a G4 frit in order to remove the lithium chloride formed in the reaction. The diethyl ether is removed in vacuo from the product solution so obtained. 811 mg of triphenylmethylium 1,2,3,4-tetraphenyl-5-pentafluorophenylcyclopentadienide are obtained.

### Example 8

Preparation of N,N-Dimethylanilinium 1,2,3,4-Tetraphenyl-5-Pentafluorophenylcyclopentadienide

2 g of 1,2,3,4-tetraphenyl-5-pentafluorophenylcyclopentadiene are dissolved in 20 ml of dried THF and reacted at −78° C. with 2.8 ml of a 1.6 molar solution of n-butyllithium in hexane. After the addition of the n-butyllithium, the reaction mixture is stirred for a further 4 hours at room temperature. The reaction mixture is then cooled to 0° C. and reacted with 0.706 g of N,N-dimethylanilinium hydrochloride. After heating to room temperature, stirring is again carried out for 4 hours. After removal of the solvent in vacuo, the crude product is taken up in 20 ml of dried diethyl ether and filtered over a G4 frit in order to remove the lithium chloride formed in the reaction. The diethyl ether is removed in vacuo from the product solution so obtained. 402 mg of N,N-dimethylanilinium 1,2,3,4-tetraphenyl-5-pentafluorophenylcyclopentadienide are obtained.

### Example 9

Preparation of Triphenylmethylium 1,2,3,4,5-Pentakis(Pentafluorophenyl)Cyclopentadienide

0.8 g of 1,2,3,4,5-pentakis(pentafluoro)phenylcyclopentadiene is dissolved in 20 ml of dried THF and reacted at −78° C. with 0.6 ml of a 1.6 molar solution of tert-butyllithium in hexane. After the addition of the tert-butyllithium, the reaction mixture is stirred for a further 4 hours at room temperature. The reaction mixture is then cooled to 0° C. and reacted with 0.24 g of triphenylmethyl chloride. After heating to room temperature, stirring is again carried out for 4 hours. After removal of the solvent in vacuo, the crude product is taken up in 20 ml of dried diethyl ether and filtered over a G4 frit in order to remove the lithium chloride formed in the reaction. The diethyl ether is removed in vacuo from the product solution so obtained. 700 mg of triphenylmethylium 1,2,3,4,5-pentakis(pentafluorophenyl)cyclopentadienide are obtained.

### Example 10

Preparation of N,N-Dimethylanilinium 1,2,3,4,5-Pentakis(Pentafluorophenyl)Cyclopentadienide

0.8 g of 1,2,3,4,5-pentakis(pentafluoro)phenylcyclopentadiene is dissolved in 20 ml of dried THF and reacted at −78° C. with 0.6 ml of a 1.6 molar solution of tert-butyllithium in hexane. After the addition of the tert-butyllithium, the reaction mixture is stirred for a further 4 hours at room temperature. The reaction mixture is then cooled to 0° C. and reacted with 0.14 g of N,N-dimethylanilinium hydrochloride. After heating to room temperature, stirring is again carried out for 4 hours. After removal of the solvent in vacuo, the crude product is taken up in 20 ml of dried diethyl ether and filtered over a G4 frit in order to remove the lithium chloride formed in the reaction. The diethyl ether is removed in vacuo from the product solution so obtained. 0.92 mg of N,N-dimethylanilinium 1,2,3,4.5-pentakis(pentafluorophenyl)cyclopentadienide is obtained.

### Example 11

Copolymerization of Ethylene and Propylene

500 ml of toluene, 4 mmol of triisobutylaluminum and 2.5 μmol of ethylenebis(tetrahydroindenyl)-zirconium dichloride were introduced into a 1.4 liter steel autoclave equipped with a mechanical stirrer, a manometer, a temperature sensor, a temperature-control device, a catalyst transfer tube and monomer metering devices for ethylene and propylene. The internal temperature was set at 40° C. by means of a thermostat. 14 g of ethylene and 33 g of propylene were then metered in. Polymerization was started by addition of 5 μmol of the compound N,N-dimethylanilinium 1,2,3,4,5-pentakis(pentafluorophenyl)cyclopentadienide. Ethylene and propylene were metered in continuously in a mass ratio of 70:30 so that the pressure at 40° C. was constant at 7 bar. After 15 minutes, a further 5 μmol of the compound N,N-dimethylanilinium 1,2,3,4,5-pentakis(pentafluorophenyl)cyclopentadienide was added. After a polymerization time of one hour, the reaction was stopped and the polymer was precipitated in methanol, isolated and dried for 20 hours at 60° C. in vacuo. There were obtained 20.8 g of a high molecular weight copolymer having the following composition: 73.2 wt. % ethylene, 26.8 wt. % propylene (determined by IR spectroscopy).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A metal- and metalloid-free cyclopentadienide compound of the general formula (II) 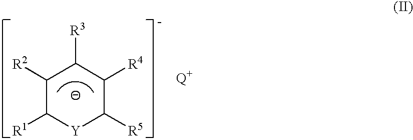
wherein
Q⁺ represents either a Lewis-acid cation according to the Lewis acid-base theory, selected from the group consisting of carbonium, oxonium and sulfonium cations, or
Q⁺ represents a Brönstedt-acid cation according to the Brönstedt acid-base theory,
Y represents a (CR₂⁶)ₘ group wherein m=from 0 to 4, wherein the radicals R⁶ may be identical or different and, when m=0, the ring may be closed or open, with the proviso that, when the ring is open, the free valences at the terminal carbon atoms are saturated by radicals R having the same meaning as R¹-R⁶,
R¹-R⁶ represent identical or different substituents selected from the group consisting of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, silyl optionally substituted by C₁-C₁₀-hydrocarbon radicals, and amine substituted by C₁- to C₂₀-hydrocarbon radicals,
with the proviso that at least one substituent is bulky.

2. A metal- and metalloid-free cyclopentadienide compound according to claim 1, wherein at least two substituents are bulky.

3. A metal- and metalloid-free cyclopentadienide compound according to claim 2, wherein at least three substituents are bulky.

4. A metal- and metalloid-free cyclopentadienide compound according to claim 1, wherein at least one R from R¹-R⁶ represents a halogen-containing aromatic compound of formula VI 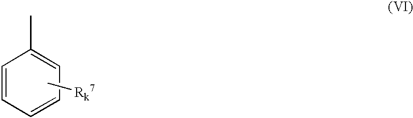
wherein
k represents an integer in the range from 1 to 5, and
R⁷ is selected from the group consisting of C₁-C₂₀-alkyl, C₁-C₂₀-alkoxy, hydrogen, halogen and C₁-C₂₅-haloalkyl, with the proviso that at least one R⁷ represents halogen or C₁-C₂₅-haloalkyl.

5. A metal- and metalloid-free cyclopentadienide compound according to claim 1, wherein at least one R from R¹-R⁶ is selected from the group consisting of 4-fluorophenyl, 4-chlorophenyl, 3-fluorophenyl, 3-chlorophenyl, 2-fluorophenyl, 2-chlorophenyl, 2,6-difluorophenyl, 2,6-di-chlorophenyl, 2,4-difluorophenyl, 2,4-dichlorophenyl, 2,3-difluorophenyl, 2,3-dichlorophenyl, 2,5-difluorophenyl, 2,5-dichlorophenyl, 3,4-difluoro-phenyl, 3,4-dichlorophenyl, 3,5-difluorophenyl, 3,5-dichlorophenyl, 2,4,6-trifluorophenyl, 3,4,5-trifluorophenyl, 2,3,4-trifluorophenyl, 2,3,5-trifluorophenyl, 2,3,6-trifluorophenyl, 2,3,4,5-tetrafluorophenyl, 2,3,5,6-tetra-fluorophenyl, 4,5,6-tetrafluorophenyl, pentafluorophenyl, 4-(trifluoromethyl)phenyl, 2,6-bis-(trifluoromethyl)phenyl, 3,5-bis-(trifluoromethyl)phenyl, 3,4,5-tris-(trifluoromethyl)phenyl, and 2,4,4-tris-(trifluoromethyl)phenyl and compounds corresponding to the compounds just mentioned in which one or more fluorine atoms have been replaced by chlorine atoms.

6. A metal- and metalloid-free cyclopentadienide compound according to claim 5, wherein at least one R from R¹-R⁶ is selected from the group consisting of 4-fluorophenyl, 2,6-difluorophenyl, 2,4-difluorophenyl, 2,4,6-trifluorophenyl, pentafluorophenyl, 3,5-bis-(trifluoromethyl)phenyl and compounds corresponding to the compounds just mentioned in which one or more fluorine atoms have been replaced by chlorine atoms.

7. A metal- and metalloid-free cyclopentadienide compound of the general formula (IIc) 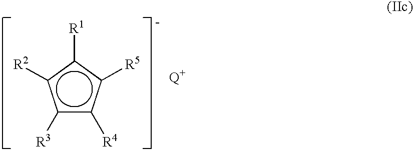
wherein
Q⁺ represents either a Lewis-acid cation according to the Lewis acid-base theory selected from the group consisting of carbonium, oxonium and sulfonium cations, or
Q⁺ represents a Brönstedt-acid cation according to the Brönstedt acid-base theory,
R¹-R⁵ represent identical or different substituents selected from the group consisting of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, sityl optionally substituted by C₁-C₁₀-hydrocarbon radicals, and amine substituted by C₁- to C₂₀-hydrocarbon radicals,
with the proviso that at least one substituent is bulky.

8. A metal- and metalloid-free cyclopentadienide compound according to claim 7, wherein at least two substituents are bulky.

9. A metal- and metalloid-free cyclopentadienide compound according to claim 8, wherein at least three substituents are bulky.

10. A process for the preparation of a metal- and metalloid-free cyclopentadienide compound of the general formula (II) 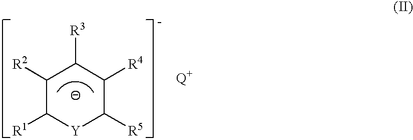
wherein
Q⁺ represents either a Lewis-acid cation according to the Lewis acid-base theory, selected from the group consisting of carbonium, oxonium and sulfonium cations, or
Q⁺ represents a Brönstedt-acid cation according to the Brönstedt acid-base theory,
Y represents a (CR₂⁶)ₘ group wherein m=from 0 to 4, wherein the radicals R⁶ may be identical or different and, when m=0, the ring may be closed or open, with the proviso that, when the ring is open, the free valences at the terminal carbon atoms are saturated by radicals R having the same meaning as R¹-R⁶,
comprising the steps of:
a) reacting a cyclopentadiene appropriately substituted by the substituents R¹-R⁶ with an alkyl metal compound or a metal to form a reaction product, wherein
R¹-R⁶ represent identical or different substituents selected from the group consisting of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, silyl optionally substituted by C₁-C₁₀-hydrocarbon radicals, amine substituted by C₁- to C₂₀-hydrocarbon radicals,
with the proviso that at least one substituent is bulky,
b) reacting the reaction product with a halide of a non-metallic cation; and
c) removing the metal halide.

11. A process according to claim 10, wherein at least two substituents are bulky.

12. A process according to claim 11, wherein at least three substituents are bulky.

13. A process for the polymerization of olefins, wherein polymerization is carried out in the presence of
a) at least one organic transition metal compound;
b) at least one cyclopentadienide compound of the general formula (II) 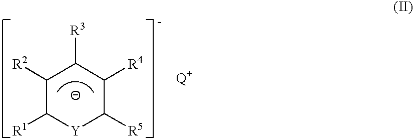
wherein
Q⁺ represents either a Lewis-acid cation according to the Lewis acid-base theory, selected from the group consisting of carbonium, oxonium and sulfonium cations, or
Q⁺ represents a Brönstedt-acid cation according to the Brönstedt acid-base theory,
Y represents a (CR₂⁶)ₘ group wherein m=from 0 to 4, wherein the radicals R⁶ may be identical or different and, when m=0, the ring may be closed or open, with the proviso that, when the ring is open, the free valences at the terminal carbon atoms are saturated by radicals R having the same meaning as R¹-R⁶,
R¹-R⁶ represent identical or different substituents selected from the group consisting of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, silyl optionally substituted by C₁-C₁₀-hydrocarbon radicals, and amine substituted by C₁- to C₂₀-hydrocarbon radicals,
with the proviso that at least one substituent is bulky, and optionally,
c) one or more organoaluminum compounds.

14. A process according to claim 13, wherein said olefins are selected from the group consisting of ethylene, propylene, but-1-ene, pent-1-ene, 4-methylpent-1-ene, hex-1-ene, oct-1-ene, isobutylene, styrene, norbornene, tetracyclododecene and unconjugated dienes.

15. A process according to claim 13, characterized in that components a) and/or b) are applied to a support.

16. A catalyst comprising a metal- and metalloid-free cyclopentadienide compound of the general formula (II) [chemical structure]
wherein
Q⁺ represents either a Lewis-acid cation according to the Lewis acid-base theory, selected from the group consisting of carbonium, oxonium and sulfonium cations, or
Q⁺ represents a Brönstedt-acid cation according to the Brönstedt acid-base theory,
Y represents a (CR₂⁶)ₘ group wherein m=from 0 to 4, wherein the radicals R⁶ may be identical or different and, when m=0, the ring may be closed or open, with the proviso that, when the ring is open, the free valences at the terminal carbon atoms are saturated by radicals R having the same meaning as R¹-R⁶,
R¹-R⁶ represent identical or different substituents selected from the group consisting of hydrogen, phenyl, aryl, C₁- to C₂₀-alkyl, C₁-C₁₀-haloalkyl, C₆-C₁₀-haloaryl, C₁- to C₁₀-alkoxy, C₆- to C₂₀-aryl, C₆- to C₁₀-aryloxy, C₂- to C₁₀-alkenyl, C₇- to C₄₀-arylalkenyl, C₂- to C₁₀-alkynyl, silyl optionally substituted by C₁-C₁₀-hydrocarbon radicals, and amine substituted by C₁- to C₂₀-hydrocarbon radicals,
with the proviso that at least one substituent is bulky.

17. A catalyst according to claim 16, wherein at least two substituents are bulky.

18. A catalyst according to claim 17, wherein at least three substituents are bulky.
